(19) United States
(12) Patent Application Publication
(10) Pub. No.: US 2024/0009336 A1
(43) Pub. Date: Jan. 11, 2024

(54) APPARATUS FOR STERILIZING MEDICAL INSTRUMENTS

(71) Applicants: Mandi Dozier, Summerville, SC (US); Kevin Rideout, Summerville, SC (US)

(72) Inventors: Mandi Dozier, Summerville, SC (US); Kevin Rideout, Summerville, SC (US)

(21) Appl. No.: 18/350,026

(22) Filed: Jul. 11, 2023

Related U.S. Application Data
(60) Provisional application No. 63/388,142, filed on Jul. 11, 2022.

(51) Int. Cl. A61L 2/18

(52) U.S. Cl. CPC A61L 2/18; A61L 2202/24; A61L 2202/122

(57) ABSTRACT

The present invention is directed to an apparatus for disinfecting a medical instrument comprising: a housing having a first end and a second end; a sponge that is adapted to be received within said housing; an entrance lid that is removably carried by said first end of said housing; an insertion opening defined in said entrance lid, said insertion opening being adapted to receive a medical instrument so that at least a portion of the medical instrument may be inserted into said insertion opening and contact said sponge; a bottom that is removably carried by said second end of said housing, wherein said bottom is adapted to store a disinfecting solution; and a transfer opening defined in said second end of said housing, whereby when said bottom is secured to said second end, said bottom is in fluid communication with said housing.

### BACKGROUND OF THE INVENTION

### (1) Field of the Invention

The present invention relates to an apparatus for decreasing or preventing acquired infections. More particularly, the present invention relates to a sterilizing device and method for decreasing or preventing acquired infections caused by medical instruments, such as suction devices.

### (2) Description of the Prior Art

Acquired infections, such as pneumonia and candidiasis in the mouth, occur when a patient develops an infection while in the hospital or a medical setting. Various devices and methods of disinfecting have been used to sterilize medical instruments, such as suction devices, with varying degrees of success. Often access to sterilization is cumbersome or awkward and only partially effective.

Of particular concern are devices such as a yankauer which is an oral suctioning tool used in medical procedures. A vakauer typically includes a firm plastic suction tip with a large opening surrounded by a bulbous head and is designed to allow effective suction without damaging surrounding tissue. This tool is used to suction oropharyngeal secretions in order to prevent aspiration. This tool is also used for patients that are on respirators for cleaning the area in and around the mouth. When not in use, such a tool can be placed somewhere in the patient's room and can be susceptible to coming in contact with viruses, bacteria or other undesirable substances.

It would be advantageous to have an object that can be used for temporary storage of such tools between uses.

It would be advantageous to have an object that can be used for preventing or eliminating undesirable materials from contacting or settling on such tools.

It would be advantageous to have an object that can be used to sterilize such tool before or between uses.

### SUMMARY OF THE INVENTION

It is an object of the present invention to provide a device for reducing and/or eliminating contamination of medical instruments

It is another object of the present invention to provide a device and method for sterilizing suction devices that is convenient and easy to use and at a low cost.

The shortcomings of the prior art are addressed by the present invention which provides a canister with a built-in sponge saturated with an anti-bacterial solution or medication for sterilizing a suction device. The canister has a screw-on storage compartment for extra solution. This storage compartment can cleanse a yankauer and suction tubing if the suction tubing gets soiled. The storage compartment can be re-useable by adding claim solution frequently throughout the day. The sterilizing device allows the medical facility to choose what type of disinfecting solution or medication preferred. Additionally, there is an attached handle that fits a variety of bedrails for patient's personal use.

In at least one embodiment, the present invention comprises: a housing having a first end and a second end; a sponge that is adapted to be received within said housing; an entrance lid that is removably carried by said first end of said housing; an insertion opening defined in said entrance lid, said insertion opening being adapted to receive a medical instrument so that at least a portion of the medical instrument may be inserted into said insertion opening and contact said sponge; a bottom that is removably carried by said second end of said housing, wherein said bottom is adapted to store a disinfecting solution; and a transfer opening defined in said second end of said housing, whereby when said bottom is secured to said second end, said bottom is in fluid communication with said housing.

In one embodiment, the apparatus further comprises a seal that is removably carried by said second end of said housing so that said seal prevents the movement of a fluid from said bottom into said housing when said seal is covering said transfer opening. In one embodiment, the seal allows the movement of a fluid from said bottom into said housing when said seal is punctured. In one embodiment, the seal is punctured by the medical instrument that has been inserted into the housing via the entrance lid. In one embodiment, the seal is punctured by insertion of the medical instrument into said transfer opening.

In one embodiment, the seal includes a first inner diameter when a medical instrument is inserted into said seal and said seal further includes a second inner diameter when the medical instrument is removed from the seal, wherein said second inner diameter is at least 50 percent smaller than said first inner diameter. In one embodiment, the first inner diameter adapted to receive a medical device so that said medical instrument may pass through said second end of said housing into said bottom and said seal further includes a flange carried by an inner wall of said valve, said flange having a second inner diameter that is smaller than said first inner diameter.

In one embodiment, the apparatus further comprises a valve that allows the medical device to pass through said transfer opening and into said bottom, wherein said valve prevents the movement of a fluid from said bottom into said housing or between said bottom and said housing when the medical device is not disposed within said valve. In one embodiment, the valve may be carried by said seal. In other embodiments, the valve may be carried by said second end of said housing.

In one embodiment said sponge includes a disinfectant and is arranged so that when the medical instrument enters said housing through said entrance lid, the medical instrument passes through an instrument receiving opening defined in a first end of said sponge and the medical device contacts said disinfectant. In one embodiment, an instrument receiving opening is defined in said first end of said sponge extends to a second end of said sponge and extends generally parallel to a longitudinal axis of said sponge.

In one embodiment, the bottom includes a first set of screw threads that are adapted to engage a second set of screw thread that is included on said second end of said housing so that said bottom can be removably secured to said housing. In at least one embodiment, the apparatus includes a handle disposed on an outer surface of said housing for carrying or positioning said housing.

Other objects, features and advantages of the present invention will become apparent from the following detailed description taken in conjunction with the accompanying drawing.

### BRIEF DESCRIPTION OF THE DRAWINGS

Having described the invention in general terms, reference will now be made to the accompanying drawings, which are not necessarily drawn to scale and wherein:

FIG. 1 a perspective, exploded view of an embodiment of the present invention;

FIG. 2 is a perspective view of an embodiment of the present invention;

FIG. 3 is a cross sectional view of the sponge that is included in an embodiment of the present invention;

FIG. 4 top plan view of the entrance lid that is included in an embodiment of the present invention;

FIG. 5A is a perspective view of one embodiment of the seal that is included in an embodiment of the present invention;

FIG. 5B is a perspective view of another embodiment of the seal that is included in an embodiment of the present invention;

FIG. 5C is a perspective view of one embodiment of the seal that is included in an embodiment of the present invention;

FIG. 6A is a top plan view of one embodiment of the seal that is included in an embodiment of the present invention;

FIG. 6B is a top plan view of another embodiment of the seal that is included in an embodiment of the present invention; and

FIG. 6C is a top plan view of another embodiment of the seal that is included in an embodiment of the present invention.

### DETAILED DESCRIPTION OF THE PREFERRED EMBODIMENT(S)

The present invention will now be described more fully hereinafter with reference to the accompanying drawings, in which preferred embodiments of the invention are shown. This invention may, however, be embodied in many different forms and should not be construed as limited to the embodiments set forth herein; these embodiments are provided so that this disclosure will be thorough and complete, and will fully convey the scope of the invention to those skilled in the art.

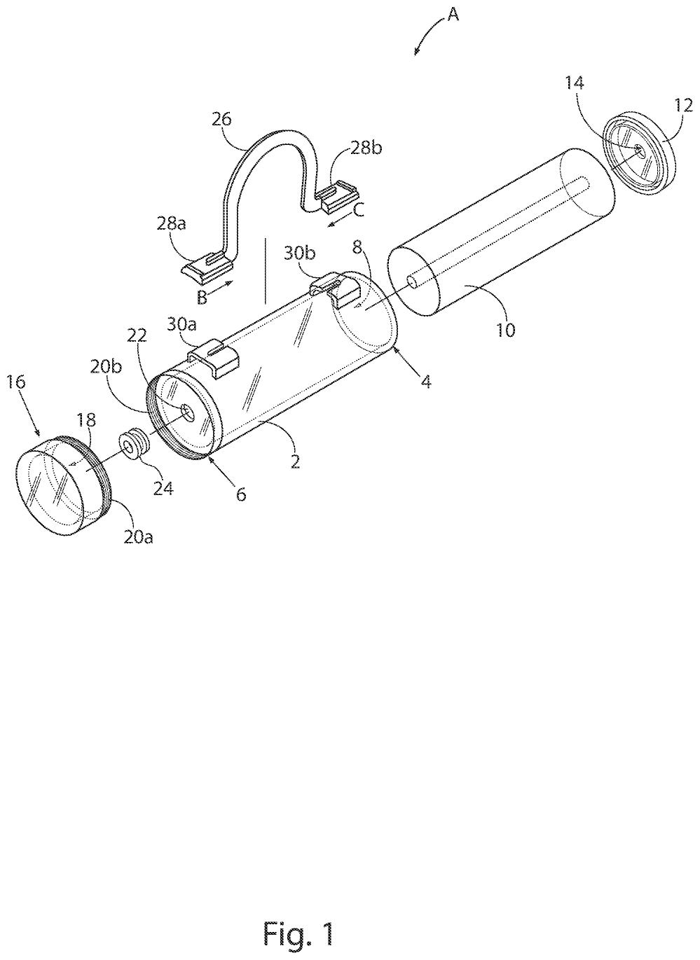

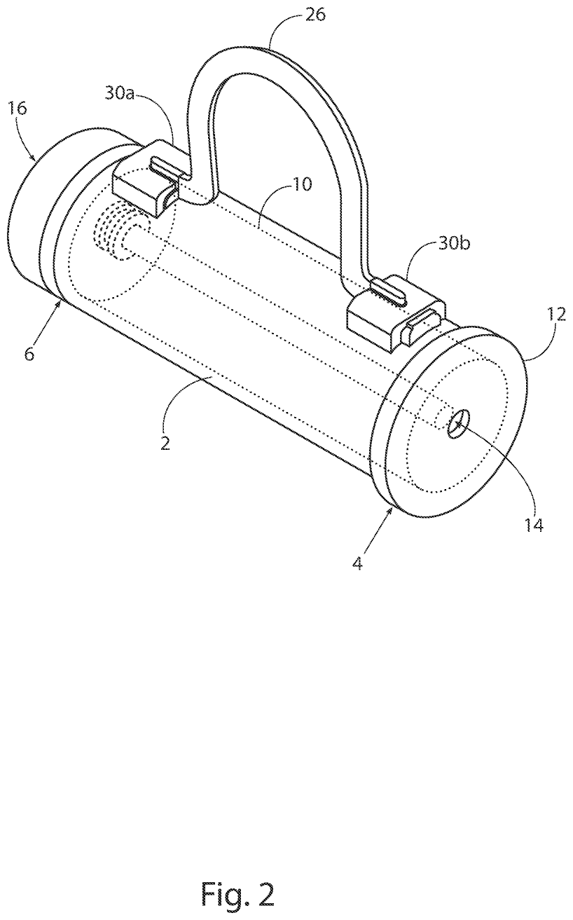

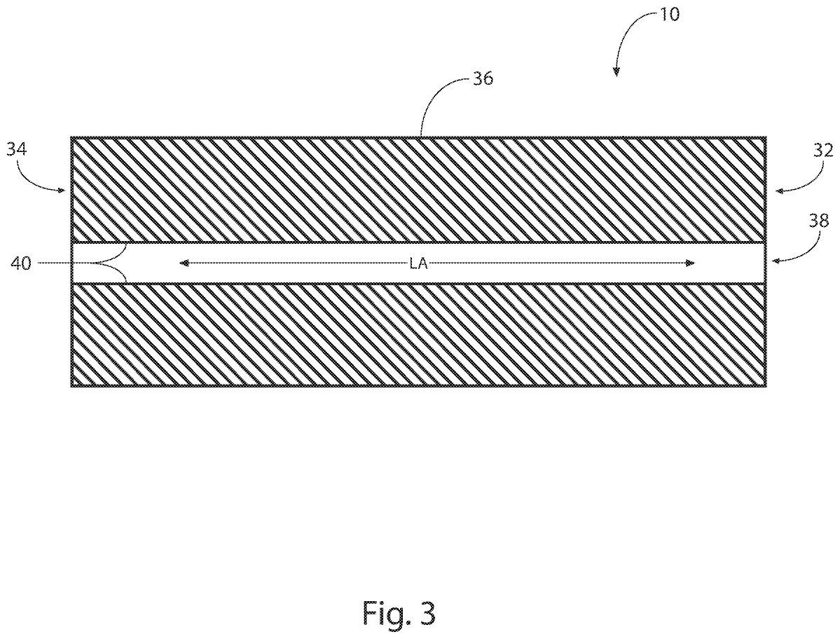

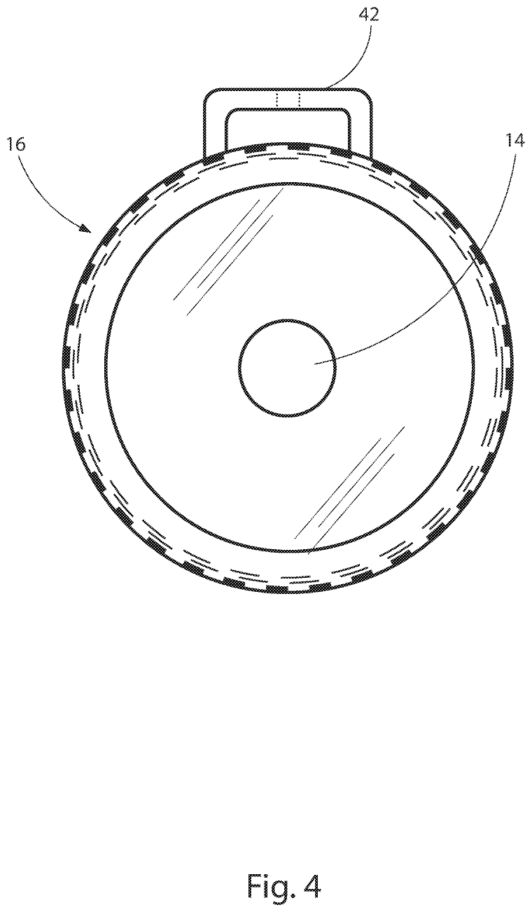

Turning now to FIGS. 1-4, there is illustrated an embodiment of the present invention showing the housing 2, which has a first end 4 and a second end 6. The housing defines an interior space 8 that is adapted to receive a disinfecting sponge 10. The housing 2 and its interior space 8 may have any cross-sectional shape. The sponge may be made of any material having absorbent properties that is generally known in the art and may also have any shape provided that it may be inserted into and/or received by the housing's interior space 8. The present invention further includes an entrance lid 12 that is adapted to be removably carried by the first end 4 of the housing. In one embodiment, the entrance lid be secured to the first end by means of screw threads. In alternate embodiments, the entrance lid may be secured by means of friction fit, compression fit, latches or any other means of removably securing a lid that are known in the art. The entrance lid may include an insertion opening 14 defined in the lid that is adapted to receive a medical device such as a yankauer or other tubing.

The present invention may further include a bottom 16 that is removably carried by the second end 6 of the housing. The bottom defines a storage compartment 18 that is adapted to receive a fluid such as a disinfectant solution. In the shown embodiment, the bottom 16 and the housing's second end 6 both include corresponding screw threads 20a and 20b that allow the bottom to be secured to housing by being screwed on. In alternate embodiments, the bottom could be removably secured by any means of securing a lid that is generally known in the art, including but not limited to compression fit, friction fit, latches and the like.

The housing 2 may further include a transfer opening 22 that is defined in the housing's second end 6. This transfer opening allows the medical device to pass from the housing's interior space 8 into the storage compartment 18 of the bottom so that it may contact the disinfectant solution contained therein. As will be discussed more fully below, in some embodiments, this transfer opening 22 also places the bottom's storage compartment 18 in fluid communication with the housing's interior space 8 and allows the disinfectant fluid to pass from the storage compartment 18 into the interior space 8 of the housing and/or between the storage compartment and the interior space.

In at least one embodiment, the housing further includes a seal 24 that is carried by the second end 6 of the housing and in some instances is disposed in or adjacent to the transfer opening 22. This seal governs when and how much disinfectant solution may be transferred between the bottom and the housing. In at least one embodiment, the seal is removably carried by the housing's second end 6 so that it can be removed for cleaning.

In the shown embodiment, the sponge is cylindrical with a circular cross-sectional shape. In other embodiments, the sponge may have other shapes. The sponge may include a first end 32, a second end 34 and an instrument receiving opening 38 that is defined in at least one of the first and second ends. In one embodiment, the instrument receiving opening 38 extends from the sponge's first end 32 to its second end 34 and does so either along or parallel to a longitudinal axis LA of the sponge. The sponge includes an outer surface 36 and an inner surface 40 that is defined by the instrument receiving opening.

In one embodiment, the present invention further includes a handle 26 that is carried by the housing 2. In one embodiment, the handle is removably carried by the housing so that the apparatus may be easily connected to a patient's bed by its railing or other framing. In the shown embodiment, the handle includes to connection tabs 28a and 28b that are adapted to be received and engaged by two corresponding securing members 30a and 30b that are carried by the outer surface of the housing 2. In the shown embodiment, the handle 26 may be secured to the housing 2 by squeezing the handle to cause the two attachment tabs 28a and 28b to move towards one another as shown by arrows B & C. Once the attachment tabs are close enough to one another, they may be inserted into the securing members 30a and 30b and the squeezing force may be stopped, allowing the attachment members to be inserted into the securing tabs. Any other method of removably attaching a handle to a housing may be used with the present invention. In one embodiment, entrance lid 16 may include a strap attachment member 42 that allows a shoulder strap to be attached thereto.

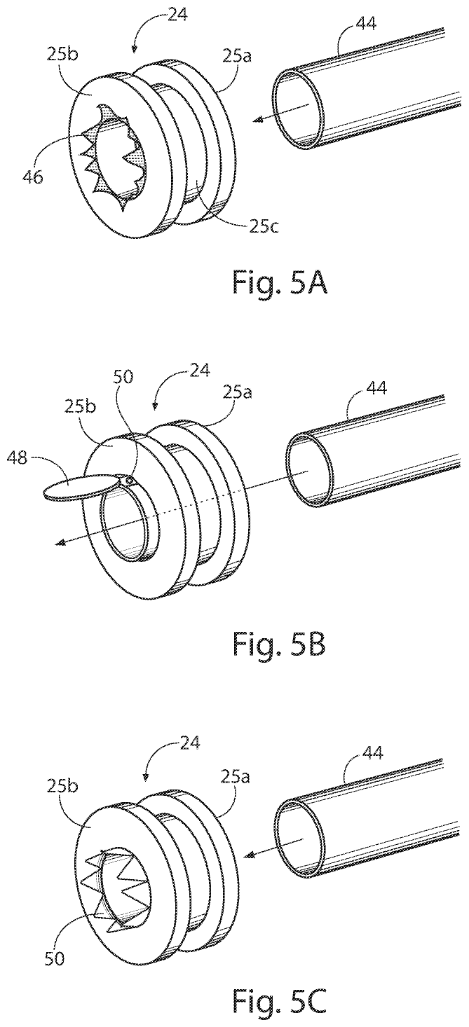

Referring now to FIGS. 5A-C and 6A-C, the seal may be more clearly seen. In the embodiment, the seal comprises a grommet that has a first end 25a and a second end 25b and a middle portion 25c so that when the seal is disposed within the transfer opening 22, the middle portion 25c passes through the transfer opening, the seal's first end 25a is located within the housing's interior space 8 and the seal's second end 25b is located within the bottom's storage compartment, thus creating a seal that prevents fluid to pass through the transfer opening 22.

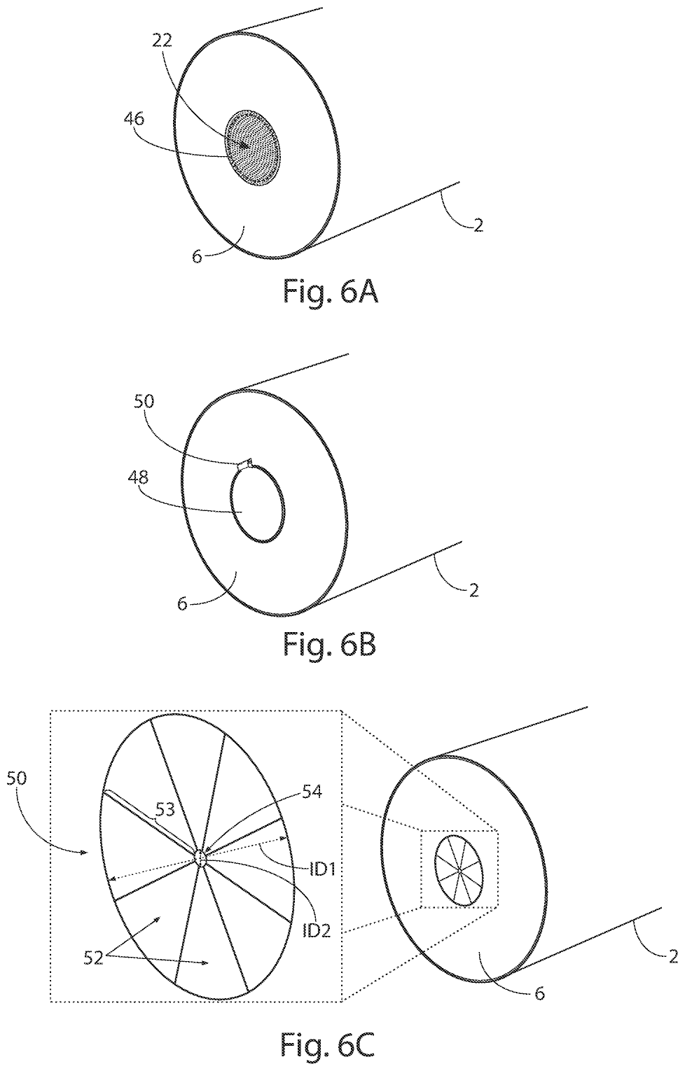

In the embodiment shown in FIGS. 5A and 6A, the seal comprises a foil 46. In one embodiment, the foil is carried by one or both of first end 25a and second end 25b of a grommet 24 that is placed in the transfer opening 22. While the seal is in a closed position (as shown in FIG. 6A), the foil prevents fluid from passing through the transfer opening. The foil 46 may be placed in an open position (shown in FIG. 5A) by being punctured or otherwise removed. In one embodiment, the foil may be placed in an open position when it is punctured by the insertion of the medical instrument 44 into and/or through the grommet 24 and/or the transfer opening 22 defined in the housing's second end 6. Once punctured, the medical instrument may pass through the transfer opening 22, into the storage compartment 18 and into the disinfectant solution contained therein. Once the foil is punctured or otherwise removed, any disinfectant solution contained in the storage compartment 18 may pass through the transfer opening and transfer between the bottom's storage compartment and the housing's interior space.

As shown in FIG. 6A, the foil 46 could be disposed directly on the second end 6 of the housing so that it covers the transfer opening. The foil could be disposed on either side of the transfer opening 22 so that the foil was generally located either inside of the housing's interior space 8 or inside of the storage compartment 18 when the bottom 16 is secured to the housing 2. The foil could be held in place by adhesive or other methods and mechanisms generally known in the art.

In the embodiment shown in FIGS. 5B and 6B, the seal 24 comprises a valve flap 48 that acts as a one-way valve. In such an embodiment, the valve flap has a closed position (shown in FIG. 6B), in which the valve flap prevents a fluid from moving in either direction between the storage compartment 18 and the interior space 8. As shown in FIG. 5B, the valve flap 48 may be placed in an open position when the medical instrument 44 is inserted through the transfer opening 22. Once the medical instrument is removed, the valve flap 48 will automatically return to the closed position. The valve flap may do so by use of a spring 50 or other methods and mechanisms of biasing a flap that are generally known in the art. In the embodiment shown in FIG. 5B, the valve flap 48 is disposed on the second end 25a of a grommet 24 that is placed into the transfer opening. As shown in FIG. 6B, however, the valve flap 48 may be disposed on the housing's second end 6, preferably on the housing's outer surface so that the flap is not inside of the interior space 8 of the housing.

In the embodiment shown in FIGS. 5C and 6C, the seal comprises a flexible membrane 50 that has an open position (FIG. 5C) and a closed position (FIG. 6C). The flexible membrane 50 may be made of any flexible material such as rubber or the like. As shown in FIG. 5C, the flexible membrane may be carried by the first end 25a of the seal; the second end 25b or both ends of a grommet 24 that is placed in the transfer opening 22. The flexible membrane may be placed in an open position when the medical instrument 44 is inserted through the grommet 24. As shown in FIG. 6C, the flexible membrane 50 may be disposed on the second end 6 of the housing such that it covers the transfer opening. In such an embodiment, the flexible membrane may be placed in an open position when the medical instrument 44 is inserted through the transfer opening 22. When in an open position, the flexible membrane 50 allows the medical instrument to pass through the transfer opening 22 disposed in the housing's second end 6 and into the storage compartment 18 of the bottom. When in an open position, flexible membrane 24 has a first inner diameter ID1, which is adapted to receive the medical instrument. Whether the flexible membrane 50 is carried by the grommet 24 or the housing's second end, the membrane's first inner diameter ID1 is preferably smaller than the diameter of the transfer opening.

When in a closed position, the flexible membrane 50 includes a small opening 54 that has a second inner diameter ID2, which is at least 50% smaller than the first inner diameter ID1 of the seal, when it is in an open position. This membrane opening 54 allows disinfectant fluid to be transferred from storage compartment 18 into the housing's interior space 8 in small quantities over time to help keep the sponge 10 moist with disinfectant solution.

In one embodiment, the flexible membrane 52 comprises a plurality of segments 52 that are preferably triangular shaped and substantially identical to one another in terms of size and dimension. These membrane segments 52 allow the membrane to smoothly transition from the closed position to the open position and vice versa. Membrane segments 52 could be created by cutting slits 53 into flexible membrane, by folding pleats into flexible membrane or by any other methods or mechanisms generally known in the art.

In use, the present invention allows for the disinfection and cleaning of a dirty medical device, including but not limited to a yankauer and/or suction tubing. The medical device is inserted through the insertion opening 14, into the instrument receiving opening 38 defined in the sponge 10 so that the medical instrument contacts the sponge's inner surface 40 so that it may be swiped with the disinfectant contained within the sponge. In some instances, the medical device includes particulate that needs to be cleaned from a surface of the medical device that does not contact the sponge. In such a case, the medical device may pass through the instrument receiving opening 38, through the transfer opening 22 and into the storage compartment 18 containing a disinfectant fluid. The disinfectant fluid may then clean all surfaces of the medical device and dislodge any particulate that is stuck thereto.

In the embodiments in which the apparatus includes a seal, the medical device transitions the seal from a closed position to an open position when the medical device passes through the transfer opening 22. In some embodiments, the seal may revert back to a closed position when the medical instrument is removed from the transfer opening, in other cases, once placed in an open position, the seal remains in that position. In at least one embodiment, the seal allows disinfectant solution to move from the storage compartment 18 into the housing's interior space 8 even when in a closed position. Preferably the transfer of fluid occurs slowly so that as the sponge begins to dry out, more disinfectant can be transferred into the housing's interior space to keep the sponge moist.

Many modifications and other embodiments of the invention set forth herein will come to mind to one skilled in the art to which these inventions pertain having the benefit of the teachings present in the foregoing descriptions. Therefore, it is to be understood that the inventions are not to be limited to the specific embodiments disclosed and that modifications and other embodiments are intended to be included within the scope of the invention. Although specific terms are employed herein, they are used in generic descriptive sense only and not for purposes of limitation.

What is claimed is:

1. An apparatus for cleaning a medical instrument comprising:
a housing having a first end and a second end;
a sponge that is adapted to be received within said housing;
an entrance lid that is removably carried by said first end of said housing;
an insertion opening defined in said entrance lid, said insertion opening being adapted to receive a medical instrument so that at least a portion of the medical instrument may be inserted into said insertion opening and contact said sponge;
a bottom that is removably carried by said second end of said housing, wherein said bottom is adapted to store a disinfecting solution; and
a transfer opening defined in said second end of said housing, whereby when said bottom is secured to said second end, said bottom is in fluid communication with said housing.

2. The apparatus of claim 1 further comprising a seal that is removably carried by said second end of said housing so that said seal prevents the movement of a fluid from said bottom into said housing when said seal is covering said transfer opening.

3. The apparatus of claim 2 wherein said seal includes a closed position and an open position, wherein said closed position inhibits the movement of a fluid between said bottom and said housing and said open position allows the movement of a fluid between said bottom into said housing.

4. The apparatus of claim 3, wherein said seal is placed in said open position when the medical instrument is inserted into said transfer opening.

5. The apparatus of claim 3, wherein said seal is placed in said open position when the medical instrument is inserted into said seal.

6. The apparatus of claim 3, wherein said seal is placed in said open position when said seal is punctured.

7. The apparatus of claim 6 wherein said seal is placed in said open position when said seal is punctured by the medical instrument that has been inserted into the housing.

8. The apparatus of claim 1 further comprising a valve flap that allows the medical device to pass through said transfer opening and into said bottom, wherein said valve flap has an opened position and a closed position, wherein said closed position prevents the movement of a fluid from said bottom into said housing.

9. The apparatus of claim 8 wherein said valve flap is placed into an open position when the medical device is inserted into said transfer opening.

10. The apparatus of claim 1 further comprising a seal carried by said second end of said housing, wherein said seal includes a first inner diameter when a medical instrument is inserted into said seal and said seal further includes a second inner diameter when the medical instrument is removed from the seal, wherein said second inner diameter is at least 50 percent smaller than said first inner diameter.

11. The apparatus of claim 1 further comprising a seal carried by said second end of said housing, wherein said seal includes having a first inner diameter adapted to receive a medical device so that said medical instrument may pass through said second end of said housing into said bottom and said seal further includes a flange carried by an inner wall of said valve, said flange having a second inner diameter that is smaller than said first inner diameter.

12. The apparatus of claim 1 wherein said sponge includes a disinfectant and is arranged so that when the medical instrument enters said housing through said entrance lid, the medical instrument passes through an instrument receiving opening defined in a first end of said sponge and the medical device contacts said disinfectant.

13. The apparatus of claim 10 wherein said instrument receiving opening defined in said first end of said sponge extends to a second end of said sponge.

14. The apparatus of claim 11 wherein instrument receiving opening defined in said first end of said sponge extends generally parallel to a longitudinal axis of said sponge.

15. The apparatus of claim 1 wherein said bottom includes a first set of screw threads that are adapted to engage a second set of screw thread that is included on said second end of said housing so that said bottom can be removably secured to said housing.

16. The apparatus of claim 1 including a handle disposed on an outer surface of said housing for carrying or positioning said housing.
